(19) United States
(12) Patent Application Publication
(10) Pub. No.: US 2016/0338566 A1
(43) Pub. Date: Nov. 24, 2016

(54) WASHING APPARATUS FOR BOTTLES AND DISHES

(71) Applicant: SHAHAR SHABTAI, PETACH TIKVA (IL)

(72) Inventor: SHAHAR SHABTAI, PETACH TIKVA (IL)

(21) Appl. No.: 15/113,840

(22) PCT Filed: Jan. 26, 2015
(86) PCT No.: PCT/IL2015/050090

§ 371 Date: Jul. 25, 2016

Related U.S. Application Data
(60) Provisional application No. 61/931,627, filed on Jan. 26, 2014.

(51) Int. Cl. A47L 15/00; A61L 2/24; A61L 2/07; A47L 15/42; B08B 3/02

(52) U.S. Cl. CPC A47L 15/0089; A47L 15/4234; A47L 15/4244; A47L 15/4287; A47L 15/4225; A47L 15/0065; B08B 3/02; A61L 2/07; A61L 2/24; A61L 2202/14; A61L 2202/23

(57) ABSTRACT

A washing apparatus including a housing, an enclosed washing space defined in the housing, a first container for clean water disposed adjacent the housing, the first container including a clean water outlet valve to selectively permit flow of water from the first container to the washing space, a second container for waste water disposed adjacent the housing, a water heater disposed in the washing space, a pump disposed in the housing and configured selectively to pump water from the washing space into the washing space and selectively to pump waste water from the washing space to the second container, a valve coupled to the pump for permitting said selective pumping of water into the washing space and said selective pumping of water to the second container; and a pre-programmed electronic controller coupled at least to the pump, the valve and the water heater to control operation at least of the first container, the pump, the valve and the water heater to wash and/or sterilize an item in the washing space.

### FIELD OF THE INVENTION

The present invention relates to the field of cleaning and sterilizing apparatus, in general, and more specifically to apparatus for washing baby bottles and small dishes.

### BACKGROUND OF THE INVENTION

Various devices are known for washing baby bottles. There are also known various devices for washing dishes. Typically, these devices are coupled to an external water source, such as a tap, and to an external drainage pipe for removing the dirty wash water during the washing cycle. These devices are not configured to sterilize the items after being washed or just sterilize the items as an independent operation.

There are also known sterilizers for baby bottles wherein the bottles are rinsed with boiling water and/or steam, or cold sterilizers which utilize chemical sterilizers. These devices do not include soap and other components for washing the bottles, as bottle sterilizers are not meant to wash milk out of the bottles. Rather, the bottles and accessories must be washed with hot, soapy water before sterilization to remove the milk residue.

Accordingly, there is a long felt need for a stand-alone washing and/or sterilizing device, and it would be very desirable if a single device could be utilized to perform either or both functions.

### SUMMARY OF THE INVENTION

There is provided according to the present invention an apparatus capable of both cleaning and sterilizing bottles, such as baby bottles, glasses and dishes. The apparatus is a stand-alone device which does not need to be connected to an external water supply. Rather, the apparatus includes a clean water container and a dirty water container and operates in a closed system. The apparatus may be modular and can include a number of additional components to aid or expand on these functions. For example, the apparatus can include a removable and/or refillable soap container, a water filter, a dispensing port for dispensing water for drinking, a tray for dry or pre-moistened wipes or towellettes, etc. Accordingly, a large variety of tasks can be performed utilizing a single apparatus, depending upon the components selected and installed by the user including, in addition to cleaning and sterilizing, providing hot water for preparing baby food or hot drinks, dispensing drinking water, and so on.

The basic apparatus for cleaning and sterilizing bottles includes a housing defining an enclosed washing space for placing at least one item for cleaning, a first removable water container for clean water and a second removable water container for waste water or water drainage. The apparatus also includes a heating element with a thermometer, thermostat or other temperature sensor, for maintaining the water at a predefined temperature according to the cleaning or sterilization process, a pump for directing water out from the washing space and circulating the water for the cleaning operation, and a sprinkler for spraying the water within the washing space throughout the cleaning and sterilizing process. Sensors, such as magnetic sensors or optical sensors, may be provided for sending an indication that the water levels in one or both containers have reached pre-determined levels, in order to control the quantity of clean water that has entered the washing space and to ensure that the waste water is disposed of before overflowing.

An electronic controller is provided for controlling the cleaning, sterilizing and all other processes of the apparatus of the present invention based on predefined programs and signals received from the various sensors and control switches. For example, the electronic controller can be programmed to operate a cleaning and sterilizing process including three stages: a first stage includes directing the water by into the washing space and heating the water to a pre-determined temperature for a predefined period; a second stage includes replacing the water in the washing space, and performing a short rinsing operation of the item in the washing space; and a third stage including directing a small quantity of water from the first container into a basin in the washing space and heating this clean water to a predefined temperature adapted for sterilizing.

There is thus provided, according to the present invention, a washing apparatus including a housing having a closable upper cover; an enclosed washing space defined in the housing; a first container for clean water disposed adjacent the housing, the first container including a clean water outlet valve to selectively permit flow of water from the first container to the washing space; a second container for waste water disposed adjacent the housing, a water heater disposed in the washing space; a pump disposed in the housing and configured selectively to pump water from the washing space back into the washing space and selectively to pump waste water from the washing space to the second container; a pump valve coupled to the pump for permitting the selective pumping of water into the washing space and the selective pumping of water to the second container; and a pre-programmed electronic controller coupled at least to the clean water outlet valve, the pump, the pump valve and the water heater to control operation at least of the clean water outlet valve, the pump, the pump valve and the water heater to wash, rinse and/or sterilize an item in the washing space.

There is also provided, according to the invention a washing apparatus including a housing having a closable upper cover; an enclosed washing space defined in the housing; a first container for clean water disposed adjacent the housing, the first container including a clean water outlet valve to selectively permit flow of water from the first container to the washing space; a second container for waste water disposed adjacent the housing, a water heater disposed in the washing space; a first pump disposed in the housing and configured to pump water from the washing space back into the washing space; a second pump disposed in the housing and coupled to the waste water container and configured to pump waste water from the washing space to the second container; and a pre-programmed electronic controller coupled at least to the clean water outlet valve, the first and second pumps and the water heater to control operation at least of the clean water outlet valve, the pumps and the water heater to wash, rinse and/or sterilize an item in the washing space.

There is further provided, according to the invention, a method for washing and/or sterilizing items in an apparatus having a housing having an upper cover and defining a washing space, a pump and a water heater disposed in the housing and an electronic controller, the method including introducing water from a clean water container coupled to the housing into the washing space; heating the water in the washing space to a pre-determined temperature for a predefined period of time; removing waste water from the washing space to a waste water container coupled to the housing; further including, before the step of removing, one action selected from the group including: pumping the water from the washing space back into the washing space; and creating steam by heating the water to a pre-determined temperature for a predefined period, so as to sterilize the items in the washing space.

### BRIEF DESCRIPTION OF THE DRAWINGS

The present invention will be more readily understood from the detailed description of embodiments thereof made in conjunction with the accompanying drawings of which:

FIG. 1 is a schematic illustration of a washing and sterilizing apparatus according to some embodiments of the invention;

FIGS. 2a and 2b are schematic illustrations of some of the washing and sterilizing apparatus components, according to some embodiments of the invention;

FIG. 3 is cross sectional view of the washing and sterilizing apparatus showing some of the components according to some embodiments of the invention;

FIG. 4 is a top sectional view of the washing and sterilizing apparatus according to some embodiments of the invention;

FIG. 5 is a block diagram illustrating the washing apparatus functional components according to some embodiments of the invention;

FIG. 6 is a flow chart illustrating the general work flow according to some embodiments of the invention;

FIGS. 7A and 7B are flow charts illustrating the washing and cleaning work flow according to some embodiments of the invention;

FIG. 8 is a flow chart illustrating the sterilizing work flow according to some embodiments of the invention; and

FIGS. 9 and 10 are schematic sectional and block diagram illustrations, respectively, of a washing and sterilizing apparatus according to alternative embodiments of the invention.

### DETAILED DESCRIPTION OF THE INVENTION

The present invention relates to an apparatus for either washing or rinsing, or sterilizing bottles or small quantities of cups and/or small dishes, or all three operations.

The apparatus is a small, compact, and cost-effective product that is intended for domestic and/or small business use. The apparatus is intended for the quick, efficient, quality and cost-effective rinsing of infant feeding bottles in particular, but may also be used for pacifiers and/or other accessories and for cups and dishes at home or in the office. The product is modular, and has several options for operation and usage. The various components can be purchased independently and the various functions can be used together with or independent from one another. With this product, the client gains a modular item that can be assembled and operated very easily, simply, quickly and safely. Furthermore, the apparatus according to an embodiment of the invention is configured to operate as a closed system without the need for coupling to an external source of water, such as a faucet, or an external point of drainage, such as a sink.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

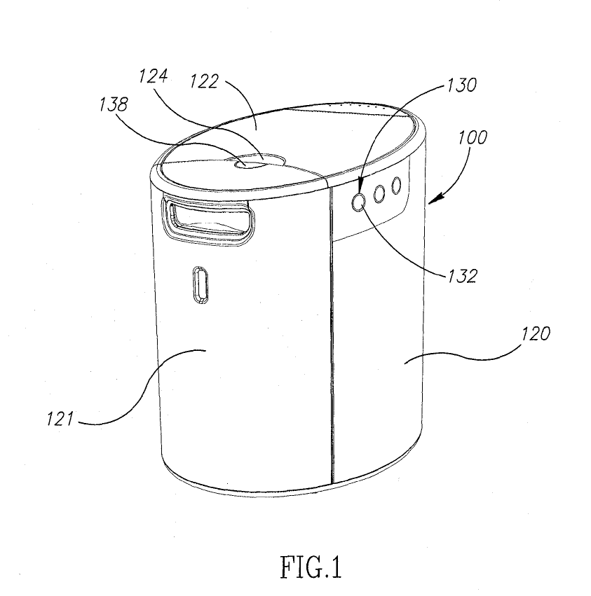

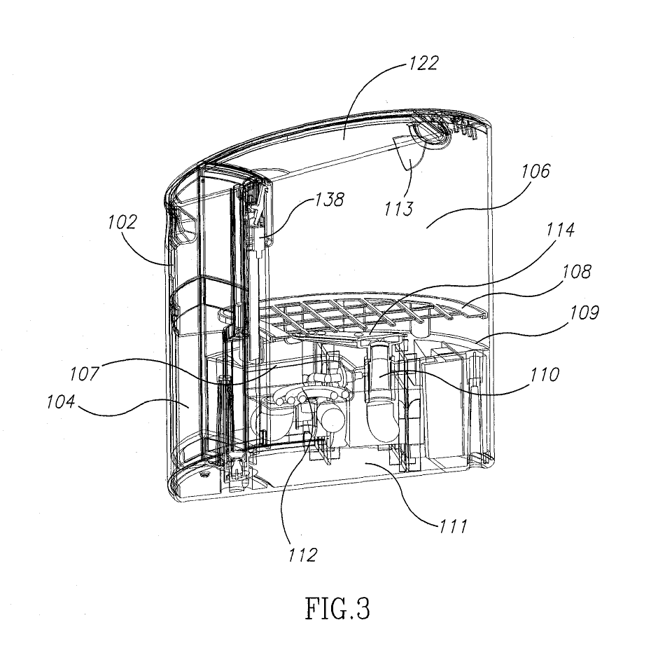

Referring now to FIG. 1, there is shown a schematic illustration of a cleaning and sterilizing apparatus 100 according to some embodiments of the invention. The figure illustrates an external view of the apparatus 100. As can be seen, apparatus 100 includes a main tank or housing 120 and a water container holder 121. Housing 120 further includes a user interface 130 including one or more selection buttons 132 enabling the user to choose the operation mode of the apparatus: cleaning mode, sterilizing mode or integrated mode of cleaning and sterilizing. Housing 120 further includes an upper cover 122. Preferably, a solenoid or otherwise electronically operated locking mechanism 138 (shown in FIG. 3), controlled by the controller, is provided to prevent inadvertent opening of the apparatus during operation while hot water is sprayed in the washing space. In addition, locking mechanism 138 permits the controller to lock and unlock and/or confirm locking of the cover 122 before beginning cleaning or sterilization. A button 124 is provided on upper cover 122 to permit a user to open the upper cover 122 and insert items to be washed into the housing. Water container holder 121 can be slid away from housing 120, so that the first and second water containers can be removed from the apparatus for filling and draining water.

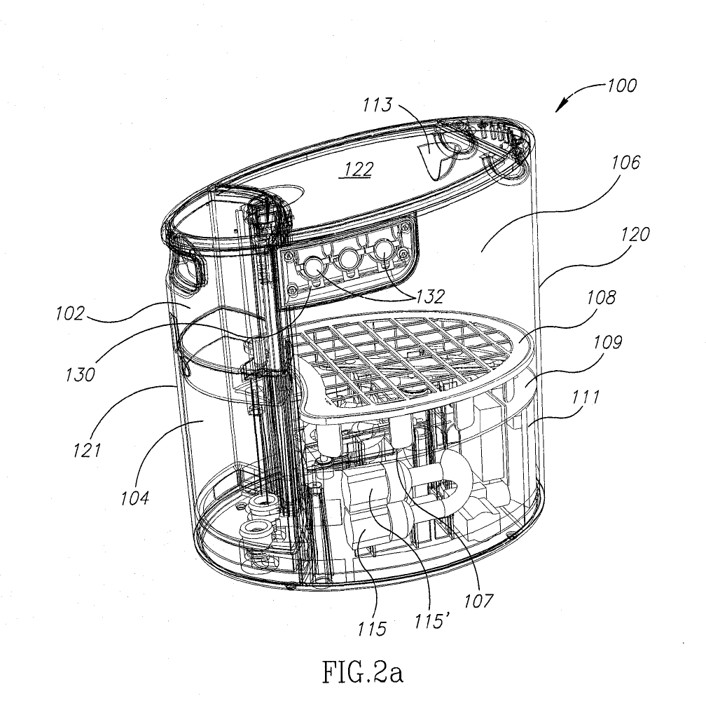

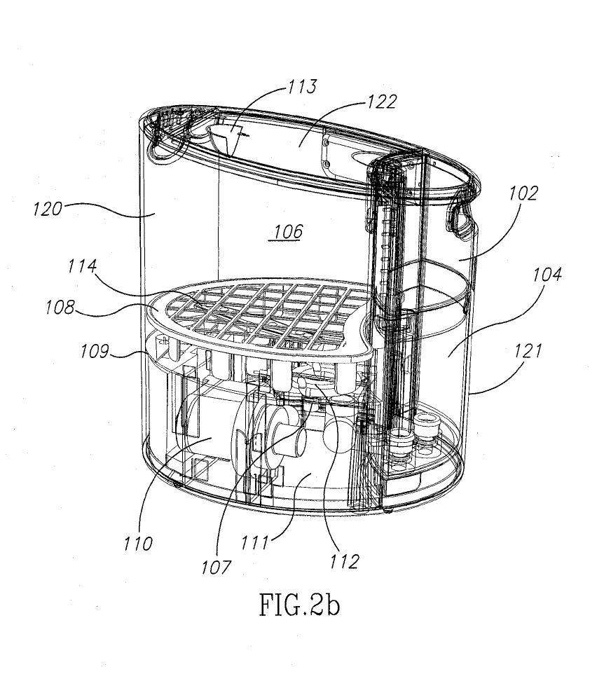

FIGS. 2a and 2b are schematic illustrations of the cleaning and sterilizing apparatus components according to some embodiments of the invention and depict an inner view of some of the main components of the apparatus. An internal partition 109 is sealingly disposed inside housing 120 and defines, in the housing, an enclosed washing space 106 and a sealed space 111 holding the various components of the apparatus. As can be seen, the washing space is defined by the walls of the housing 120, the partition 109 and the upper cover 122. Preferably, the washing space is a sealed space for improved operation when steam is created in the washing space.

Partition 109 includes a basin 107 for holding water, in which a heating element 112 is disposed. A perforated washing surface 108, on which items to be washed can be placed, is mounted in washing space 106 on, and spaced apart from, partition 109. Washing surface 108 can be any surface having a plurality of holes to permit washing water and steam to pass therethrough. A holder or dispenser 113 is located inside the upper cover 122 for holding and dispensing a cleansing material during the washing stage of operation.

A first container 102 for clean water and a second container 104 for waste water are disposed adjacent housing 120. In the illustrated embodiment, first and second containers 102 and 104 are disposed one above the other so as to be removable from the apparatus for filling, emptying and cleaning. First container 102 includes an electronic water outlet valve (not shown) to permit the controller to control the outflow of water from the container to the washing space in the housing. Second container 104 includes a water inlet having an electric waste valve 115 for receiving waste water from the washing space. One-way valves are preferably provided, as known, to permit or prevent water from entering or leaving the first and second containers when they are not disposed in the proper alignment in the washing apparatus.

In the illustrated embodiment, first and second containers 102 and 104 are disposed adjacent washing space 106, with first container 102 disposed higher than partition 109, to permit water from first container 102 to flow by gravity into the washing space 106, when the controller opens the outlet valve. Alternatively, the first and second containers can be disposed above, below or to the side of the washing space, as desired.

The apparatus further includes a water pump 110. Water pump 110 is arranged to circulate water in the washing space, as described in detail below. Water pump 110 is also arranged to receive waste water from the washing space 106 and pump it into the second container 104. Preferably, an electronic controller is disposed in the sealed space 111, or on or adjacent housing 120, or in a drawer (not shown) under the housing 120, and is programmed to control the work flows of the washing, rinsing and sterilizing operations and the electric components of the apparatus. The apparatus preferably is connected to the mains electricity, to a battery, or to any other suitable power source. It will be appreciated that, if desired, a second water pump can be provided for pumping clean water from the first container into the washing space.

FIG. 3 is a cross sectional view of the cleaning and sterilizing apparatus components according to some embodiments of the invention from a different angle than FIG. 2. Like elements have like reference numerals. FIG. 3 also shows the first container for the clean water 102, the second container for waste water, the washing space 106, the washing surface 108 and pump 110. In FIG. 3, the heating element 112 for heating the washing water can best be seen, disposed in basin 107 in partition 109. Heating element 112 is coupled to the controller, so as to be activated to heat any water in the basin and in the washing space according to the pre-programmed cleaning and/or sterilizing schedule.

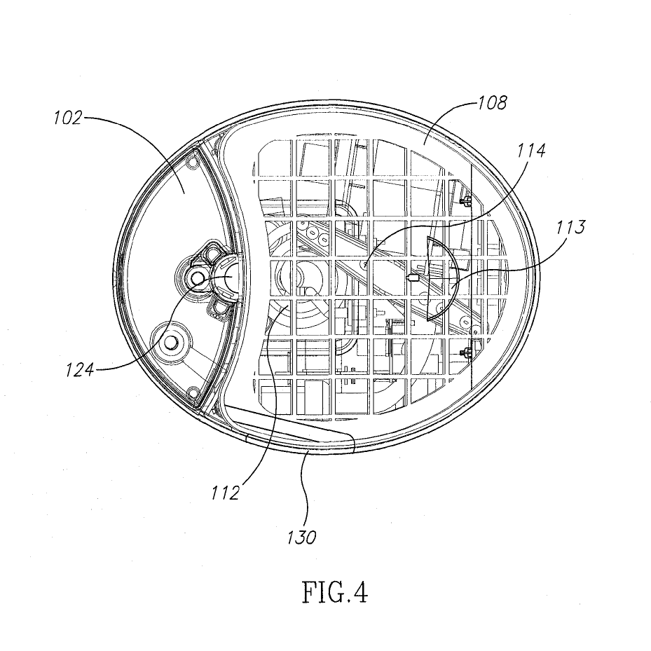

FIG. 4 is an upper sectional view of the cleaning and sterilizing apparatus components according to some embodiments of the invention. In the illustration of FIG. 4, a sprinkler 114 disposed in housing 120 can be seen. Sprinkler 114 is coupled to the pump 110 via an electric circulation valve 115′, which provides pressurized clean water from the basin to the sprinkler so the sprinkler can spray that water upwards in jets throughout the washing space 106. Preferably, the sprinkler is arranged to spray water that has been heated in the basin when the heating element is energized.

According to some embodiments of the invention, a drawer (not shown) is provided underneath the housing. The electronic controller can be disposed in the drawer to provide additional sealing protection against water contacting the controller.

It will be appreciated that FIGS. 1 to 4 are schematic illustrations and part of the piping system has been left out of the drawings to simplify the drawings and clarify the positioning of the components by not obscuring the view.

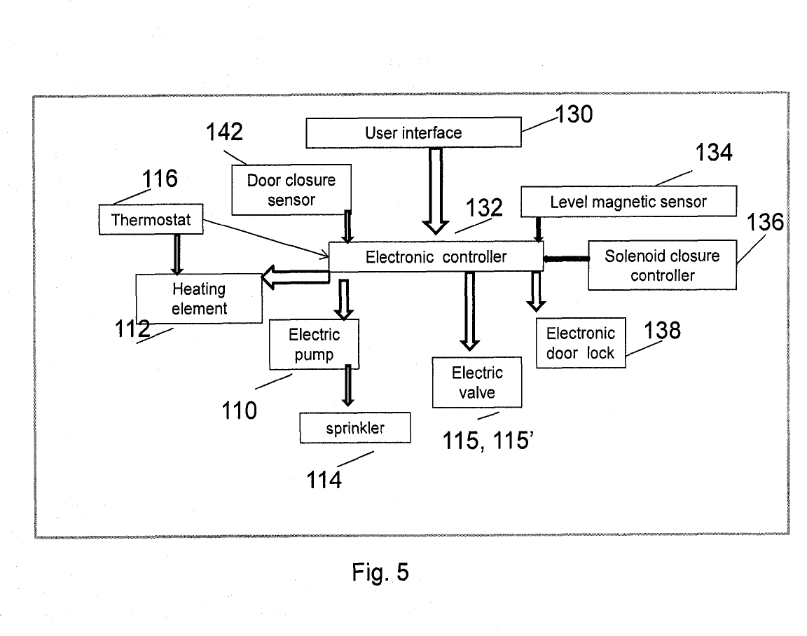

As stated above, the apparatus of the present invention can be used as a washer, as a sterilizer, or as both. FIG. 5 is a block diagram of the functional components of the cleaning and/or sterilizing apparatus according to some embodiments of the invention. The cleaning and/or sterilizing apparatus, as illustrated, includes an electronic controller 132 which is programmed to control the work flows of the washing, rinsing and sterilizing operations. The electronic controller 132 receives user input of the selected operating mode, such as via the user interface selection button 132, and the measurements of various sensors disposed throughout the apparatus. These sensors can include, for example, at least one of: an upper cover closure sensor 142, a thermostat 116, a water level sensor 134 (e.g., magnetic, optic) associated with each water container, and a solenoid closure controller 138 for upper cover 122. Based on the measurements of the sensors, the controller validates the initial conditions for safe operation. During the working process of the apparatus, the controller regulates the operation of whichever of the following components are present in the system: the heating element 112, the electric pump 110, the sprinkler 114, circulation and waste valves 115, 115′, and the solenoid closure controller 136 of the optional electronic upper cover lock 138. The regulation of the operation mode is based on predefined programs and pre-selected sensor indications.

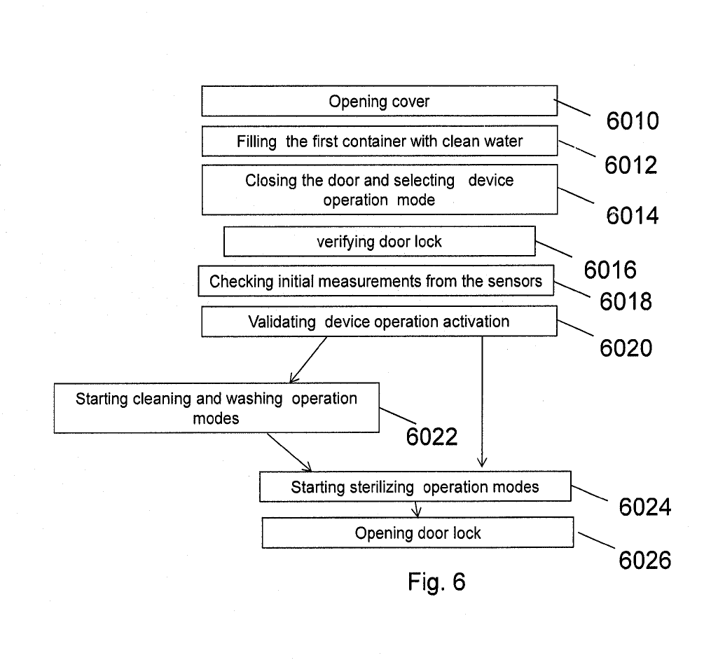

FIG. 6 is flow chart illustrating the general work flow according to some embodiments of the invention. Prior to operation, the user opens the upper cover 122 (step 6010) and places the items to be washed on the washing surface 108. In the illustrated embodiment, at this time, soap or detergent or other cleansing material, is placed in holder 113 in the cover. Before activating the apparatus, the user opens the container holders to remove the first water container and fill it with clean water (step 6012). If necessary, the user also removes the second water container to empty out the waste water.

After closing the upper cover, which causes any cleansing material in the holder in the cover to spill into the washing space, the user selects the operation mode: washing and rinsing only, sterilizing only or combined operation of cleaning, cleaning and sterilizing (step 6014). Once the controller confirms that the upper cover is closed and locked (step 6016), the controller checks whether pre-selected initial operating conditions, such as water level in the container or temperature, are met (step 6018). If the pre-set initial operating conditions are met, the controller validates activation of the device (step 6020). According to the user selection, the appropriate operation mode is activated: washing (cleaning) and rinsing (washing) (step 6022), or sterilizing (step 6024). When the washing and/or sterilization process has been completed, the door lock is opened and the items can be removed (step 6026). It will be appreciated that, when the upper cover is locked by a locking solenoid 138, the controller ensures that the upper cover cannot be opened by the user before the operation of cleaning or sterilizing has been completed. Upon completion of the operation, the controller unlocks the cover lock so the user can open it and remove the sterilized items.

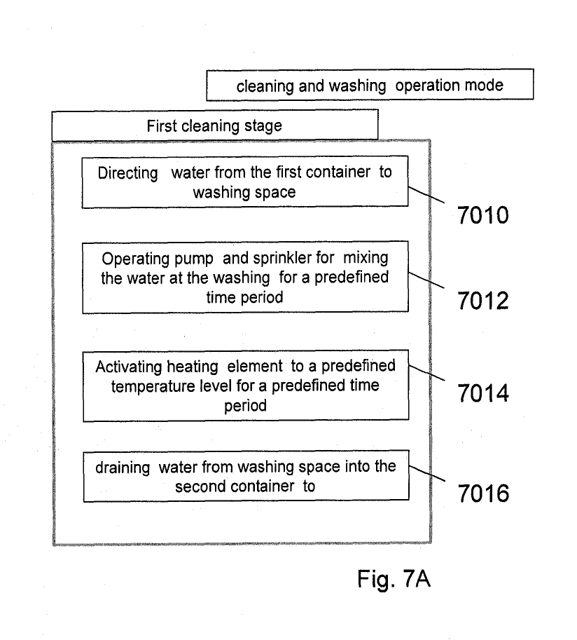

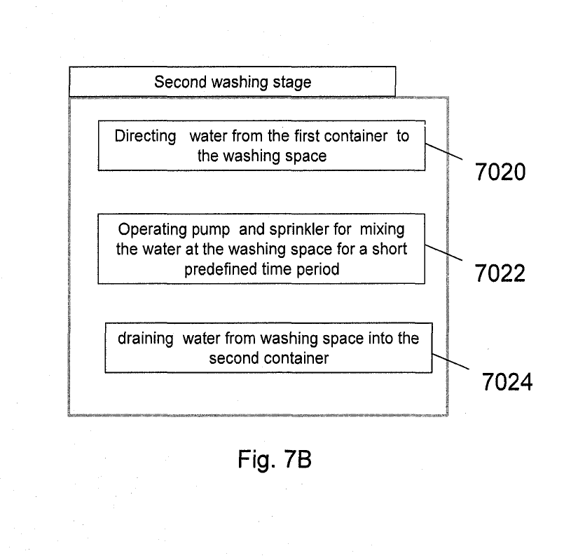

FIGS. 7A and 7B are flow charts illustrating the washing and rinsing work flow according to some embodiments of the invention. During the first cleaning stage, illustrated in FIG. 7A, the clean water is directed from the first water container to the washing space (step 7010) by the controller opening the clean water outlet valve to permit water flow by gravitation, or by operating a pump (not shown in the drawings). When the water level sensor in the clean water container sends a signal indicating that a pre-defined quantity of water has flowed from the container into the washing space, the controller sends a signal to close the water outlet valve in the first container.

During the cleaning phase, the pump pumps water from the basin to the sprinkler, so that the water in the washing space is pumped back into the washing space throughout the washing and rinsing stages. This circulation of the water causes the water to mix with any cleansing material in the washing space, and the mixture is sprayed throughout the washing space for a pre-defined period of time (step 7012). At the same time, the heating element is activated to heat the water to a predefined temperature, for example 65° C., or any other pre-selected temperature, and maintain the water at that temperature for a pre-defined period of time (step 7014). Thus, when the desired water temperature is reached, or the water heater reaches the desired temperature, the thermometer, thermostat, or other temperature sensor, signals the controller that the water temperature or water heater has reached the pre-defined level. The controller then sends a signal to open circulation valve 115′ and to activate the pump, to permit the water to be pumped from the basin 107 to the sprinkler 114. The cleaning phase is programmed to continue for a pre-defined time period, with the sprayed water falling back onto the partition 109 and into the basin 107. In this way, the heated water is sprayed and distributed throughout the washing space by means of the sprinkler and the pump maintains the water in circulation, as the items are sprayed with hot water and cleansing material several times during the washing operation during the pre-defined period of time. At the end of this period, the waste water is drained from the washing space into the second container (step 7016). This can be accomplished by closing circulation valve 115′ and opening waste valve 115 to permit the pump to pump the waste water from the basin into the waste water container.

During the second washing stage, for rinsing the washed items, illustrated in FIG. 7B, the same steps are repeated using only clean water with no additional materials for a pre-defined time period, typically shorter than the washing time period, in order to rinse the washed items in the apparatus. Thus, the clean water is directed from the first water container to the washing space (step 7020) by the controller opening the clean water outlet valve to permit water flow by gravitation, or by operating a pump (not shown in the drawings). When the water level sensor in the clean water container sends a signal indicating that a pre-defined quantity of water has flowed from the container into the washing space, the controller sends a signal to close the water outlet valve in the first container.

During the rinsing phase, the heating element may, but need not, be activated to maintain a predefined temperature of the water. During the rinsing phase also, the pump pumps water from the basin to the sprinkler. At the same time, if desired, the heating element is activated to heat the water to a predefined temperature and maintain the water at that temperature during rinsing. Thus, when the desired water temperature is reached, if the water is heated, the thermostat, or other temperature sensor, signals the controller that the water temperature has reached the pre-defined level. Otherwise, this step is not necessary. The controller then sends a signal to open circulation valve 115′ and to activate the pump, to permit the water to be pumped from the basin 107 to the sprinkler 114. The pump and sprinkler are operated so as to spray and circulate the clean water throughout the washing space for a pre-defined period of time (step 7022) in order to rinse off the items in the washing space. The rinsing phase also is programmed to continue for a pre-defined time period, with the sprayed water falling back onto the partition 109 and into the basin 107. In this way, the rinse water is sprayed and distributed throughout the washing space by means of the sprinkler and the pump maintains the water in circulation, as the items are sprayed with rinse water several times during the rinsing operation during the pre-defined period of time. At the end of the pre-defined time period, the waste water is drained from the washing space into the second container (step 7024). This can be accomplished by closing circulation valve 115′ and opening waste valve 115 to permit the pump to pump the waste water from the basin into the waste water container.

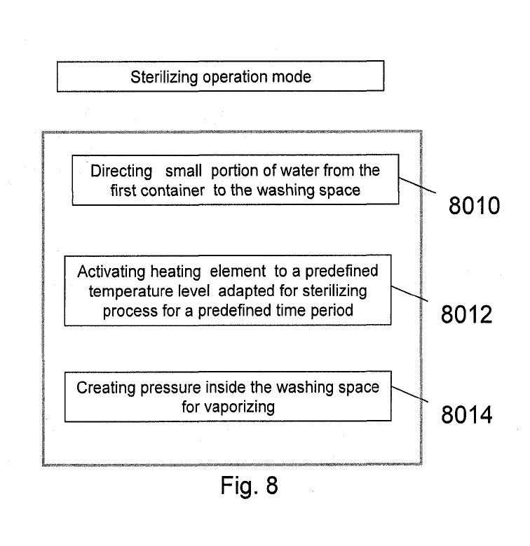

FIG. 8 is flow chart illustrating the sterilizing work flow according to some embodiments of the invention. In the first stage of the sterilizing process, a pre-defined quantity of water, typically less than used for washing or rinsing, is directed from the first container to the washing space (step 8010) in the same way as described for the washing stage. When the desired water level is reached, the water level sensor signals the controller that the water level has reached the pre-defined level. In response, the controller sends a signal to activate the heating element to heat this water to a predefined temperature, for example 100° C., to boil the water and create steam for sterilizing the items in the apparatus for a pre-defined period of time (step 8012). A thermostat or other temperature sensor signals the controller when the heating element reaches a pre-defined temperature so the controller can start and stop energizing the heater to maintain the desired temperature. As stated above, it is desirable that the apparatus be configured with a completely sealed washing space so as to permit pressure to rise in the washing space during the sterilization phase. The pressure inside the washing space is adapted for creating vapor, by maintaining the temperature of the heating element and continuing to generate steam (step 8014). The sterilizing phase is programmed for a pre-defined time period, in accordance with sanitizing requirements.

It will be appreciated that, while operation of the apparatus has been described above with regard to a sensor in the clean water container signaling the controller when a pre-determined quantity of water has flowed into the washing space so the controller starts operation, alternatively, the controller can automatically start each operation after a pre-programmed period of time has lapsed.

According to alternative embodiments of the invention, a separate pump can be provided for circulating the water inside the washing space during the washing and rinsing operations, and a separate pump for pumping out the waste water to the waste water container. In this case, two separate electric valves are not required. Rather, one pump can be connected directly to the sprinkler and the second pump can be connected directly to the waste container, and the controller merely switches between the pumps.

It will be appreciated that the programming of the various operations can be fixed in the factory. Alternatively, the controller can be programmable and can be re-programmed by a user according to his or her specific requirements or desires.

It will be appreciated that this method of operation, with two internal water containers, creates an independent, stand-alone washing and sterilizing device that does not require any coupling to the main water supply. Thus, the apparatus can easily be placed on a counter in a kitchen or office.

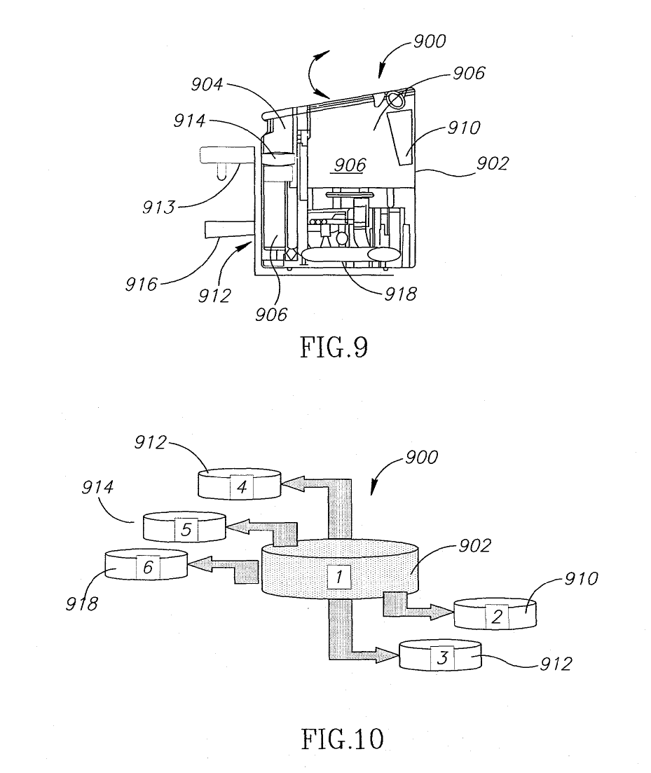

FIGS. 9 and 10 are schematic illustrations of a washing and sterilizing apparatus 900 constructed and operative according to alternative embodiments of the invention. Apparatus 900 is a small, compact modular system for domestic and/or office use, for cleaning and rinsing bottles and/or dishes and/or small accessories.

Apparatus 900 includes a housing 902, a clean water tank 904, a waste water tank 906, and a washing space, here illustrated as a main rinsing and cleaning container 906. In addition, this apparatus is a modular apparatus which can be used in a full and inclusive manner by purchasing and attaching all of its components and accessories, or it can be purchased and/or assembled with only a few of its component parts. The following components and accessories may be added on an individual basis to the apparatus housing 902:

A. A removable soap or detergent holder or dispenser 910 to be disposed inside the housing.

B. A front head 912 including a faucet or drinking water dispenser 913 mounted outside of the housing, for pouring hot or cold water, coupled by a water transduction pipe to the clean water tank.

C. A water filter 914, to filter water before dispensing it to the drinking water dispenser, to be disposed inside the housing.

D. A heated area or platform 916 on front head 912 to hold and dry washed items and/or for placing items, such as a bottle or a cup, to be heated before preparing a hot drink or to maintain their heat.

E. A drawer 918 to hold dry or pre-moistened towelettes, or any other desired items.

In other words, the apparatus for cleaning infant feeding bottles and/or dishes and/or accessories according to these alternative embodiments has a sophisticated and broad business model that allows for the purchase of a modular product in stages and/or in full. In addition, the apparatus allows the consumption of various perishable components. This model therefore creates a connection and a dependency between the seller of the product and the client, one that is based on different profit focuses: Purchasing the basic product for cleaning feeding bottles and/accessories;—Purchasing an additional part or parts for the product and/or a full set of all its other components; Purchasing perishable components including: soap dispenser, cleansing material, water filter, pouring faucet, heated platform, and a towelette dispenser and towelettes.

It will be appreciated from the description above that the apparatus of some embodiments of the present invention involves the integration of several technologies, systems and various functions in order to enable an optimal, practical and available usage system for the rinsing, cleaning and sterilization of infant bottles and/or dishes and/or various different accessories.

The apparatus may include a modular machine that has the capacities to perform a varied number of cleaning, rinsing, sterilization and heating actions, and/or the preparation of food and/or beverages for infants and/or children and/or adults and/or elderly people.

In particular, the apparatus for rinsing bottles and/or dishes has a simple design using a technique with a very small and limited level of mechanical operation. In addition, the apparatus permits the usage of a liquid and/or powder and/or crystallized soap, which is consumed after a limited use.

Accordingly, users of the apparatus according to these last embodiments—for example, mothers, fathers and/or any other consumer who is interested in preparing beverages and/or washing dishes before and/or after use—obtain with this product the possibility of a complete set of actions and usages concerning the preparation of food and/or beverage bottles for infants prior to preparation and/or after use, including the preparation of coffee/tea and/or any other beverage in a domestic and/or office environment.

The apparatus according to the invention cleans, rinses and sterilizes dishes/bottles and/or other tools and so can have three basic states for water temperature (cold, lukewarm/normal, hot). A solution providing all three options creates a significant advantage in a single product, for it significantly increases the usage options and variety of consumers that may purchase and use the product. In addition, the apparatus detailed here can be made from and/or utilize environmentally-friendly and/or recycled materials and/or aid in the protection of the environment and the Earth, including by saving water, one of the most important natural resources.

It is a product which can be used with a minimum mechanical operation, and with a limited number of components and/or accessories that are added according to the user's requirements—in order to clean and rinse infant feeding bottles and/or other dishes. It is a machine or device for rinsing infant feeding bottles and/or other dishes, which may include a front head with a faucet for pouring hot and/or cold water, combined with a platform for heating bottles and/or drinking cups, as well as an apparatus for pre-moistened or dry towelettes, according to the user's preference.

While the invention has been described with respect to a limited number of embodiments, it will be appreciated that many variations, modifications and other applications of the invention may be made. It will further be appreciated that the invention is not limited to what has been described hereinabove merely by way of example. Rather, the invention is limited solely by the claims which follow.

1. A washing apparatus comprising:
a housing having a closable upper cover;
an enclosed washing space defined in the housing;
a first container for clean water disposed adjacent the housing, the first container including a clean water outlet valve to selectively permit flow of water from the first container to the washing space;
a second container for waste water disposed adjacent the housing,
a water heater disposed in the washing space;
a pump disposed in the housing and configured selectively to pump water from the washing space back into the washing space and selectively to pump waste water from the washing space to the second container;
a pump valve coupled to the pump for permitting said selective pumping of water into the washing space and said selective pumping of water to the second container; and
a pre-programmed electronic controller coupled at least to the clean water outlet valve, the pump, the pump valve and the water heater to control operation at least of the clean water outlet valve, the pump, the pump valve and the water heater to wash, rinse and/or sterilize an item in the washing space.

2. A washing apparatus comprising:
a housing having a closable upper cover;
an enclosed washing space defined in the housing;
a first container for clean water disposed adjacent the housing, the first container including a clean water outlet valve to selectively permit flow of water from the first container to the washing space;
a second container for waste water disposed adjacent the housing,
a water heater disposed in the washing space;
a first pump disposed in the housing and configured to pump water from the washing space back into the washing space;
a second pump disposed in the housing and coupled to the waste water container and configured to pump waste water from the washing space to the second container; and
a pre-programmed electronic controller coupled at least to the clean water outlet valve, the first and second pumps and the water heater to control operation at least of the clean water outlet valve, the pumps and the water heater to wash, rinse and/or sterilize an item in the washing space.

3. The washing apparatus according to claim 1, further comprising a partition defining a basin, the water heater being disposed in the basin, the washing space being defined by the partition, interior walls of the housing, and the upper cover.

4. The washing apparatus according to claim 1, further comprising a perforated washing surface disposed in the washing space for holding items to be washed.

5. The washing apparatus according to claim 1, further comprising an additional pump for pumping clean water from the first container to the washing space.

6. The washing apparatus according to claim 1, further comprising a sprinkler coupled to the pump and arranged to spray jets of water from the pump into the washing space.

7. The washing apparatus according to claim 1, further comprising at least one sensor selected from the group including:
a sensor for sensing a quantity of water in the clean water container and providing a signal corresponding thereto to the controller;
a sensor for sensing a quantity of water in the waste water container and providing a signal corresponding thereto to the controller;
a temperature sensor sensing temperature of the heating element and providing a signal corresponding thereto to the controller;
a temperature sensor sensing temperature of the water and providing a signal corresponding thereto to the controller;
an upper cover sensor sensing whether an upper cover of the housing is closed and providing a signal corresponding thereto to the controller.

8. The washing apparatus according to claim 1, further comprising an upper cover electric locking mechanism for locking the upper cover of the housing.

9. The washing apparatus according to claim 1, further comprising a drawer disposed under the housing for holding the electronic controller.

10. The washing apparatus according to claim 1, further comprising at least one modular component selected from the group including:
a removable soap or detergent holder or dispenser to be disposed inside the housing;
a front head including a faucet or drinking water dispenser mounted outside the housing coupled by a water transduction pipe to the clean water container;
a water filter, to filter water before dispensing it to the drinking water dispenser, to be disposed inside the housing.
a heated platform on a front head to hold and dry washed items and/or for placing items, such as a bottle or a cup, to be heated before preparing a hot drink or to maintain their heat; and
a drawer to hold selected items disposed outside of the housing.

11. A method for washing and/or sterilizing items in an apparatus having a housing having an upper cover and defining a washing space, a pump and a water heater disposed in the housing and an electronic controller, the method comprising:
a. introducing water from a clean water container coupled to the housing into the washing space;
b. heating the water in the washing space to a pre-determined temperature for a predefined period of time;
c. removing waste water from the washing space to a waste water container coupled to the housing;
further comprising, before the step of removing, one action selected from the group comprising:
d. pumping the water from the washing space back into the washing space; and
e. creating steam by heating the water to a pre-determined temperature for a predefined period, so as to sterilize the items in the washing space.

12. The method according to claim 11, wherein the step of pumping further includes:
introducing a cleansing material into the washing space; and
circulating a mixture of the water and the cleansing material within the washing space.

13. The method according to claim 11, further comprising:
b. introducing a cleansing material into the washing space after the step of introducing clean water;
d. pumping a mixture of the water and the cleansing material from the washing space back into the washing space to wash items in the washing space before the step of removing;
f. introducing more clean water from the clean water container into the washing space;
g. pumping the water from the washing space back into the washing space to rinse items in the washing space;
h. removing the water from the washing space to the waste water container;
i. introducing more clean water from the clean water container into the washing space; and
j. creating steam by heating the water to a pre-determined temperature for a predefined period, so as to sterilize items in the washing space.

14. The method according to claim 11, further comprising:
sensing a water level in the clean water container and sending a signal to the controller when a pre-defined water level is reached to stop introducing clean water, after each said step of introducing clean water.

15. The method according to claim 11, further comprising:
sensing a temperature of a water heater or the water in the washing space and sending a signal to the controller to stop heating the water, when a pre-defined temperature is reached, after each said step of heating water.

16. The washing apparatus according to claim 2, further comprising a partition defining a basin, the water heater being disposed in the basin, the washing space being defined by the partition, interior walls of the housing, and the upper cover.

17. The washing apparatus according to claim 2, further comprising an additional pump for pumping clean water from the first container to the washing space.

18. The washing apparatus according to claim 2, further comprising at least one sensor selected from the group including:
a sensor for sensing a quantity of water in the clean water container and providing a signal corresponding thereto to the controller;
a sensor for sensing a quantity of water in the waste water container and providing a signal corresponding thereto to the controller;
a temperature sensor sensing temperature of the heating element and providing a signal corresponding thereto to the controller;
a temperature sensor sensing temperature of the water and providing a signal corresponding thereto to the controller;
an upper cover sensor sensing whether an upper cover of the housing is closed and providing a signal corresponding thereto to the controller.

19. The method according to claim 13, further comprising:
sensing a water level in the clean water container and sending a signal to the controller when a pre-defined water level is reached to stop introducing clean water, after each said step of introducing clean water.

20. The method according to claim 13, further comprising:
sensing a temperature of a water heater or the water in the washing space and sending a signal to the controller to stop heating the water, when a pre-defined temperature is reached, after each said step of heating water.
